(19) United States
(12) Patent Application Publication
(10) Pub. No.: US 2002/0052414 A1
(43) Pub. Date: May 2, 2002

(54) Use, in cosmetic preparations, of prostaglandin EP-2 and/or EP-4 receptor antagonists to attenuate, reduce or stop the growth of head hair and other hairs

(76) Inventors: Bruno Bernard, Neuilly-sur-Seine (FR); Jean Francois Michelet, Creteil (FR); Yann Mahe, Morsang-sur-Orne (FR)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 09/917,211

(22) Filed: Jul. 30, 2001

(30) Foreign Application Priority Data
Jul. 28, 2000 (FR) 00 09986

(51) Int. Cl. A61K 31/557; A61K 7/00

(52) U.S. Cl. 514/559; 514/573; 424/401

(57) ABSTRACT

The present invention relates to a use, in cosmetic preparations, of prostaglandin EP-2 and/or EP-4 receptor antagonists to attenuate, reduce or stop the growth of head hair and other hairs, and to the cosmetic treatment process using these compositions.

The present invention relates to a use, in cosmetic preparations, of prostaglandin EP-2 and/or EP-4 receptor antagonists to attenuate, reduce or stop the growth of head hair and other hairs, and to the cosmetic treatment process using these compositions.

It appears important in many uses such as depilation, in order to combat hirsutism and unwanted hairs or to prevent hair growth on a bald head to have available compounds for preparing compositions for attenuating, reducing or stopping the growth of keratin materials.

Patent WO 96/09806 by Handelman describes compositions for reducing hair growth, containing a sufficient amount of protein kinase C inhibitor such as verapamil, thioridazine, curcumin, etc.

Moreover, the document “VII. International Union of Pharmacology Classification of Prostanoid Receptors: Properties, Distribution, and Structure of the Receptors and Their Subtypes” by Coleman et al., Pharmacological Reviews, 1994, Vol. 46 mentions the existence of prostaglandin EP-2 and/or EP-4 receptor antagonists.

The document “Growth regulation of primary human keratinocytes by prostaglandin E receptor EP-2 and EP-3 subtypes” by Konger et al., (Biochimica Biophysica Acta 1401, 1998, 221-224) reveals that prostaglandin EP-2 and/or EP-4 receptors are involved in regulating epithelial cell growth.

Nevertheless, it is well known that the programmes of differentiation of the keratinocytes of the epidermis and of hair follicles are clearly different. Thus, it is known that differentiation markers such as keratins K1 and K10 are not expressed in hair follicles and in particular in the outer sheath (Lenoir et al., 1988, Dev. Biol. 130: 610-620); that trichohyalin is expressed in hair follicles, in particular in the inner sheath but not in the epidermis (O'Guin et al., 1992, J. Invest. Dermatol. 98: 24-32); and that type 1 cyclooxygenase is not expressed in the keratinocytes of hair follicles but is expressed in the epidermis (Michelet et al., 1997, J. Invest. Dermatol. 108: 205-209).

Furthermore, it is known that the keratinocytes of the epidermis and of hair follicles behave differently in response to the same pharmacological agent. Thus, it is known that, in vivo, treating the epidermis with retinoic acid induces hyperplasia and spongiosis (Griffiths et al., 1993, J. Invest. Dermatol. 101: 325-328) whereas treating the scalp induces a loss of hair (Berth-Jones et al., 1990, Br. J. Dermatol. 122: 751-755), and that, in vitro, retinoic acid, depending on the dose used, promotes or reduces the differentiation of the epidermis (Asselineau et al., 1989, Dev. Biol. 133: 32-335), while it causes an interruption of growth of the hair follicles (Billoni et al., 1997, Acta Dermatol. Venerol. 77: 350-355). It is also known that EGF induces epidermal hyperplasia and, simultaneously, regression of the hair follicles (Philip et al., 1985, J. Invest. Dermatol. 84: 172-175).

The Applicant has discovered, entirely surprisingly, that it is possible to limit the growth of head hair or other hairs by using prostaglandin EP-2 and/or EP-4 receptor antagonists.

The Applicant has thus found that the use in accordance with the invention produces a rapid effect and makes it possible to use a prostaglandin EP-2 and/or EP-4 receptor antagonist at a low concentration and/or with a low rate of application. These agonists are capable of attenuating, reducing or stopping the growth of head hair and other hairs and/or of increasing the loss of head hair and other hairs.

These compounds are particularly non-toxic and show good storage properties.

The use of these compounds makes it possible to obtain, in particular compared with those known previously, more effective compositions which may be used in a particularly simple manner, and which also allow easy removal of the compositions by simple rinsing.

The compositions in accordance with the invention are moreover particularly suitable in cosmetic and dermatological terms and do not cause any irritation of the scalp, even after prolonged contact, without rinsing.

One subject of the present invention relates to the use of prostaglandin EP-2 and/or EP-4 receptor antagonists to attenuate, reduce or stop the growth of head hair and other hairs, as cosmetic and dermatological agents.

Another subject of the invention consists of the use of prostaglandin EP-3 receptor agonists to prepare a cosmetic or dermatological composition.

Another subject of the invention consists of the cosmetic or dermatological process for treating head hair and other hairs using such a composition.

Other subjects of the invention will become apparent on reading the description and the examples which follow.

The use of prostaglandin EP-2 and/or EP-4 receptor antagonists in accordance with the invention makes it possible to attenuate, reduce or stop the growth of head hair and other hairs. These prostaglandin EP-2 and/or EP-4 receptor antagonists act as cosmetic or dermatological agents for attenuating, reducing or stopping the growth of head hair and other hairs. These agonists are also capable of attenuating, reducing or stopping the growth of head hair and other hairs.

Prostaglandins are biological effectors derived from polyunsaturated fatty acids such as, for example, arachidonic acid for PGA₂, PGE₂, PGF_{2α}, and TXA₂, or dihomo-γ-linolenic acid for PGE₁. Prostaglandins are involved in many physiological regulation phenomena.

The prostaglandin EP-2 and EP-4 receptors are receptors of the EP receptor family which comprises a very large number of different receptors when the large variations in the tissue activity of the prostaglandins is considered.

An antagonist is a compound which binds to a receptor and which does not induce a biological response, in contrast with an agonist, which induces a biological response similar to that obtained with the natural ligand which activates this response. It may moreover prevent or reduce the efficacy of the natural ligand which normally activates this response.

The term “other hairs” also means head hair, the eyelashes, the eyebrows and all hairs in general, whatever their anatomical location.

The preferred compounds are chosen more particularly from AH6809 and AH23848B.

Compound AH6809 is described in the article “Prostanoid Receptors: Structures, Properties, and Functions” by Shuh Narumya et al., Physiological Review, 1999, 1203-1204.

Compound AH23848B is described in the article “Identification of prostaglandin E receptor “EP-2” cloned from mastocytoma cells as EP4 subtype”, FEBS Lett, 364, 339-341, 1995.

Another subject of the present invention is the use of prostaglandin EP-2 and/or EP-4 receptor antagonists in or for the preparation of a cosmetic or dermatological composition for topical application, for attenuating, reducing or stopping the growth of head hair and other hairs.

This cosmetic composition may contain from 0.001% to 10% and preferably from 0.1% to 5% of antagonists by weight relative to the weight of the composition.

In accordance with the invention, the cosmetic compositions may also contain prostaglandin EP-3 receptor agonists, such as sulprostone, compound TEI3356, compound M&B28767 or prostaglandin PGE1, which are described in the article “Prostanoid Receptors: Structures, Properties, and Functions” by Shuh Narumya et al., Physiological Review, 1999, 1203-1204, in order to increase the effect of losing the head hair and other hairs.

The cosmetically or dermatologically acceptable medium for the compositions according to the invention consists more particularly of water and optionally of cosmetically acceptable organic solvent.

The organic solvents may represent from 5% to 98% of the total weight of the composition. They may be chosen from the group consisting of hydrophilic organic solvents, lipophilic organic solvents and amphiphilic solvents, or mixtures thereof.

Thus, the composition may also contain a cosmetically acceptable medium consisting of water or water and at least one organic solvent chosen from the group consisting of hydrophilic organic solvents, lipophilic organic solvents and amphiphilic solvents, or mixtures thereof.

Among the hydrophilic organic solvents which may be mentioned, for example, are linear or branched lower monoalcohols containing from 1 to 8 carbon atoms, for instance ethanol, propanol, butanol, isopropanol, isobutanol; polyethylene glycols having from 6 to 50 ethylene oxides; polyols such as propylene glycol, isoprene glycol, butylene glycol, glycerol or sorbitol; monoalkyl or dialkyl isosorbides in which the alkyl groups contain from 1 to 5 carbon atoms, for instance dimethyl isosorbide; glycol ethers, for instance diethylene glycol monomethyl or monoethyl ether and propylene glycol ethers, for instance dipropylene glycol methyl ether.

Amphiphilic organic solvents which may be mentioned are polyols such as propylene glycol (PPG) derivatives, such as fatty acid esters of polypropylene glycol, PPG ethers of fatty alcohols, for instance PPG-23 oleyl ether, and PPG-36 oleate.

Lipophilic organic solvents which may be mentioned, for example, are fatty esters such as diisopropyl adipate, dioctyl adipate and alkyl benzoates.

The preferred organic solvents are chosen from the group consisting of monofunctional or polyfunctional alcohols, optionally oxyethylenated polyethylene glycols, polypropylene glycol esters, sorbitol and its derivatives, dialkyl isosorbides, glycol ethers, polypropylene glycol ethers and fatty esters.

In order for the cosmetic or dermatological compositions of the invention to be more pleasant to use (milder on application, more nourishing and more emollient), it is possible to add a fatty phase to the medium for these compositions.

The fatty phase preferably represents from 0% to 50% of the total weight of the composition.

This fatty phase may comprise one or more oils preferably chosen from the group consisting of:

volatile or non-volatile, linear, branched or cyclic, organomodified or unmodified, water-soluble or liposoluble silicones,

mineral oils such as liquid paraffin and liquid petroleum jelly,

oils of animal origin such as perhydrosqualene,

oils of plant origin such as sweet almond oil, avocado oil, castor oil, olive oil, jojoba oil, sesame oil, groundnut oil, macadamia oil, grapeseed oil, rapeseed oil or coconut oil,

synthetic oils such as purcellin oil and isoparaffins,

fluoro oils and perfluoro oils,

fatty acid esters such as purcellin oil.

It may also comprise as fatty substances one or more fatty alcohols, fatty acids (ferric acid) or waxes (paraffin, polyethylene wax, carnauba wax or beeswax).

The composition may also additionally contain at least one additive chosen from the group consisting of conventional hydrophilic or lipophilic gelling agents and/or thickeners; hydrophilic or lipophilic active agents; preserving agents; antioxidants; fragrances; emulsifiers; moisturizers; pigmenting agents; depigmenting agents; keratolytic agents; vitamins; emollients; sequestering agents; surfactants; polymers; acidifying or basifying agents; fillers; free-radical scavengers; ceramides; sunscreens; insect repellents; slimming agents; colorants; bactericides; anti-dandruff agents.

The amounts of these various adjuvants are those used conventionally in the fields under consideration.

Needless to say, a person skilled in the art will take care to select the optional compound(s) to be added to the composition according to the invention such that the advantageous properties intrinsically associated with the composition in accordance with the invention are not, or are not substantially, adversely affected by the addition envisaged.

The compositions according to the invention may be in any presentation form normally used for topical application, in particular in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules, these spherules possibly being polymer nanoparticles such as nanospheres and nanocapsules, or better still lipid vesicles of ionic and/or nonionic type.

The compositions may have the appearance of a white or coloured cream, an ointment, a milk, a lotion, a serum, a paste, a mousse or a solid.

They may optionally be applied to the skin in the form of an aerosol.

They may also be in solid form, and for example in the form of a stick. They may be used as care products and/or as make-up products.

The composition may have a pH of between 3 and 8.

Another subject of the present invention consists of a cosmetic or dermatological treatment process for attenuating, reducing or stopping the growth of head hair and other hairs, which consists in applying to the head hair and/or other hairs a cosmetically or dermatologically effective amount of prostaglandin EP-2 and/or EP-4 receptor antagonists.

According to another subject of the invention, the cosmetic or dermatological treatment process for attenuating, reducing or stopping the growth of head hair and other hairs also consists in applying to the head hair and/or other hairs a cosmetic or dermatological composition as defined.

The examples which follow are intended to illustrate the invention without, however, being limiting in nature:

### EXAMPLES OF LOTIONS FOR PREVENTING HAIR GROWTH

### Example I

| | | |
|---|---|---|
| AH6809 | 0.03 | g |
| Propylene glycol | 20 | g |
| 95° ethanol | 30 | g |
| Water | qs 100 | g |

This lotion is applied daily at a rate of 10 ml to the scalp, for 2 to 3 months. A marked reduction in the daily growth of head hair and other hairs is then observed.

### Example II

| | | |
|---|---|---|
| AH23848B | 0.15 | g |
| Polyglyceryl 3-hydroxylauryl ether | 26 | g A.M. |
| Hydroxypropylcellulose sold under the | 2 | g |
| name Klucell G by the company Hercules | | |
| Preserving agent | qs | |
| 95° ethanol | 50 | g |
| Water | qs 100 | g |

This lotion is used daily as a shampoo at a rate of 15 g per head of hair, with an exposure time of about one minute, for a period of 4 months. An appreciable reduction in the daily growth of the hair is then observed.

### Example III

| | | |
|---|---|---|
| AH6809 | 0.3 | g |
| Propylene glycol | 20 | g |
| 95° ethanol | 30 | g |
| Water | qs 100 | g |

This lotion is applied daily at a rate of 10 ml to the scalp, for 2 to 3 months. A marked reduction in the daily growth of head hair and other hairs is then observed.

### Example IV

| | | |
|---|---|---|
| AH23848B | 0.15 | g |
| Polyglyceryl 3-hydroxylauryl ether | 26 | g A.M. |
| Hydroxypropylcellulose sold under the | 2 | g |
| name Klucell G by the company Hercules | | |
| Preserving agent | qs | |
| Sulprostone | 1 | g |
| 95° ethanol | 50 | g |
| Water | qs 100 | g |

This lotion is used daily as a shampoo at a rate of 15 g per head of hair, with an exposure time of about one minute, for a period of 4 months. An appreciable reduction in the daily growth of the hair is then observed.

Experiment

In order to study the behaviour of hair follicles in the presence of prostaglandin EP-2 and/or EP-4 receptor antagonists, the Applicant used the “surviving hair” method from L'Oréal Patent FR 9508465.

From a scalp biopsy, a fairly thin strip of scalp was isolated using a scalpel. With microtweezers, the adipose tissue around the follicles was removed, while taking care not to damage the hair bulb. Under a microscope, the follicle was cut away using a scalpel to separate it from its epidermal and dermal environment.

One of the fragments obtained was cultured in Williams E medium at 37° C. under a humid atmosphere in the presence of 5% CO₂ and was used as control.

The other fragments were placed in the same culture medium in the presence of prostaglandin EP-2 and/or EP-4 receptor antagonists: AH6809 and AH23848B.

The fragments in the presence of the agonists thus maintained in histoculture shorten in a significantly greater manner in comparison with the antagonist-free control fragment.

1. Use of prostaglandin EP-2 and/or EP-4 receptor antagonists as cosmetic agents for attenuating, reducing or stopping the growth of head hair and other hairs.

2. Use according to claim 1, characterized in that the antagonists are chosen from AH6809 and AH23848B.

3. Use of prostaglandin EP-2 and/or EP-4 receptor antagonists according to claim 1 or 2, in or for the preparation of a cosmetic composition for topical application, for attenuating, reducing or stopping the growth of head hair and other hairs.

4. Use according to claim 3, characterized in that the said cosmetic composition contains from 0.001% to 10% and preferably from 0.1% to 5% of antagonists by weight relative to the weight of the composition.

5. Use according to claim 3 or 4, characterized in that the cosmetic compositions may also contain prostaglandin EP-3 receptor agonists, in order to increase the loss of head hair and other hairs.

6. Use according to any one of claims 3 to 5, characterized in that the composition also contains a cosmetically acceptable medium consisting of water or water and at least one organic solvent chosen from the group consisting of hydrophilic organic solvents, lipophilic organic solvents and amphiphilic solvents, or mixtures thereof.

7. Use according to claim 6, characterized in that the organic solvents are chosen from the group consisting of monofunctional or polyfunctional alcohols, optionally oxyethylenated polyethylene glycols, polypropylene glycol esters, sorbitol and its derivatives, dialkyl isosorbides, glycol ethers, polypropylene glycol ethers and fatty esters.

8. Use according to claim 6 or 7, characterized in that the organic solvent(s) represent(s) from 5% to 98% of the total weight of the composition.

9. Use according to any one of claims 3 to 8, characterized in that the composition also comprises at least one fatty phase.

10. Use according to claim 9, characterized in that the fatty phase represents from 0% to 50% of the total weight of the composition.

11. Use according to any one of claims 3 to 10, characterized in that the composition contains at least one additive chosen from the group consisting of conventional hydrophilic or lipophilic gelling agents and/or thickeners; hydrophilic or lipophilic active agents; preserving agents; antioxidants; fragrances; emulsifiers; moisturizers; pigmenting agents; depigmenting agents; keratolytic agents; vitamins; emollients; sequestering agents; surfactants; polymers; acidifying or basifying agents; fillers; free-radical scavengers; ceramides; sunscreens; insect repellents; slimming agents; colorants; bactericides; anti-dandruff agents.

12. Use according to any one of claims 3 to 11, characterized in that the composition is in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules.

13. Use according to any one of claims 3 to 12, characterized in that the composition has the appearance of a white or coloured cream, an ointment, a milk, a lotion, a serum, a paste, a mousse or a solid.

14. Use according to any one of claims 3 to 13, characterized in that the composition has a pH of between 3 and 8.

15. Cosmetic treatment process for attenuating, reducing or stopping the growth of head hair and other hairs, characterized in that it consists in applying to the head hair and other hairs a cosmetically effective amount of antagonists as defined in either of claims 1 and 2.

16. Cosmetic treatment process for attenuating, reducing or stopping the growth of head hair and other hairs, characterized in that it consists in applying to the head hair and other hairs a cosmetic composition as defined in any one of claims 3 to 14.
